(19) United States
(12) Patent Application Publication
(10) Pub. No.: US 2009/0215723 A1
(43) Pub. Date: Aug. 27, 2009

(54) Organic silicic acids complex for therapeutic and cosmetic applications

(76) Inventor: Tien Cahn Le, Montreal (CA)

Correspondence Address: DAVID S. RESNICK, NIXON PEABODY LLP, 100 SUMMER STREET, BOSTON, MA, 02110-2131, US

(21) Appl. No.: 11/912,220

(22) PCT Filed: Apr. 20, 2006
(86) PCT No.: PCT/CA06/00643

§ 371 Date: May 23, 2008

Related U.S. Application Data
(60) Provisional application No. 60/672,874, filed on Apr. 20, 2005.

(51) Int. Cl. A61K 31/695; C07F 7/02; A61P 1/00

(52) U.S. Cl. 514/63; 556/466

(57) ABSTRACT

This invention describes materials containing organic silica, used in cosmetic for external uses (gel, cream or lotion) or for internal uses (microbeads/beads, tablets or solutions). These materials are vegetable extracts such as bamboo or algae which constitute richest sources in organic silica (more soluble and assimilable). After extraction in a basic medium, the organic silica extract can form a stable complex at acidic pH in presence of stabilizers such as gelatin, collagen, polyethylene glycol and/or chitosan, etc. These complexes can be used for cosmetic applications in gel, lotion and cream or as a matrix in order to immobilize various bioactive molecules. For internal uses, the soluble form of monomeric or oligomeric silicic acid is necessary to preserve its assimilability. In this context, the addition of stabilizers (gelatin and/or collagen) or the functionalization of silicic acid is possible to improve its solubility and biodisponibility.

### CROSS-REFERENCE TO RELATED APPLICATIONS

This application claims the benefit of U.S. provisional application 60/672,874 filed Apr. 20, 2005 which is incorporated herein by reference in its entirety.

### TECHNICAL FIELD

The invention relates to organic silicic acid complexes and derivatives for therapeutic and cosmetic use, and especially relates to organic silicic acid complexes and derivatives having increased aqueous stability.

### BACKGROUND OF THE INVENTION

Silicon is the most abundant element in the earth's crust after oxygen and it is always in a combination state (never native). It is often found in the form associated with aluminum, from where its name, SIAL. For the inorganic forms, they are represented mainly in silica (SiO₂) and silicates forms. Its abundance and particular properties makes silicon a valuable mineral.

It is also found in a vitreous form (silica glass), a hydrated silica, presenting itself in gel form or of colloidal solution (according to the concentration of the aqueous solution) or an amorphous silica, dehydrated gel at moderate temperature.

The oxygenated derivatives of silicon have an acid character; the SiO₂ is an anhydride acid. The orthosilicic acid, Si(OH)₄, a dissolution form of silica, is a very weak acid (at 25° C., pK₁=9.8 and pK₂=12). This acid can thus react with metallic ions only with a pH lower than the precipitation pH of the corresponding hydroxide acid. This allows the bonding formation with iron and aluminum (exclude the Ca²⁺ and the Mg²⁺ at physiological pH of 7.4). These complex compounds are most probably involved in the metabolic biochemical phenomena.

Si(OH)₄ is slightly soluble in pure water and above pH 9.0, the silicic acid is ionized and its solubility increases quickly. In contrast, its solubility decreases in presence of cations such as calcium, aluminum or iron.

Anhydrous silica is ten times less soluble than amorphous hydrated silica (10 mg/l). Beyond these limiting concentrations, tetrahedrons have the tendency to form silica gel (polymerization form). In practice, the silicon atom is almost always associated to one (or several) atom (atoms) of oxygen. However, the possibility of direct bonding with nitrogen (Si—N) gave to the silatranes.

The hydrolysis of —Si—O—Si— bonding in silica (and the mineral silicates) eventually releases the silicic acid in solution and it is in this form that silica enters the biosphere.

The biological interaction that should draw more attention is the particular reactivity of the silicic acid. Thus, the silicic acid reacts directly with the amine groups of proteins and the phosphate ester groups of phospholipids. The silicic acid is known to react with all the membrane systems and can induce significant changes of permeability.

The association of silica to organic materials starts to be apprehended. The hydroxylated surface of silica gels supports the captation of several polar organic molecules, giving rise to their use in chromatography. The polyhydroxylated compounds in the biological systems (particularly polysaccharides) interact with silica gels by hydrogen interactions.

The chemistry of silicic acid and its polymers is dominated by the following characteristics:
The polymerization capacity of silicic acid to form dimers, trimers and tetramers, etc. giving polysilicic acid (hydrated amorphous silica). The polymerization is maximal at pH of 5.5 and strongly influenced by the presence of polar organic molecules;
The capacity of the silicic acid and its polymers to associate with polar organic molecules by hydrogen interactions;
The reactivity of silanol groups with carboxylic compounds to form esters:

—Si—OH+HOOC—R→—Si—OOC—R+H₂O
Si(OH)₄ is known to react with the cis-diols, particularly with catechols, polyphenols and salicylic acid, etc.

Silicon is one of the major trace elements entering in the organism composition and plays a fundamental biological role. Silicon can, in fact, present itself in two different forms: mineral and organic.

Mineral silicon is widespread in a natural environment in various forms: quartz, sand, clays, rocks, stones (topazes). Mineral silicon comes from the diatomite, rock formed in the ocean floors by algae and river sands. It can be solid (e.g. dioxide of silicon) or liquid (orthosilicilic acid).

Organic silicon differs from mineral silicon by the adjacent presence of at least a carbon atom related to hydrogen, such as CH₃ Si(OH)₃. Organic silicon constitutes an assimilable source of silicon by the organism which elaborates from sand or plants, etc. It is worth to mention that the organic silicon cannot exist in a natural state, because of its strong affinity with the oxygen molecules.

In the organism, silicon is mainly present in bones, tendons, muscles, blood vessel walls, liver, spleen, heart, thyroid, kidneys and thymus. Silicon constitutes a major element of the organism support structures: skin, vessels, bone and cartilaginous system.

### Food Contributions

Cereals and food fibers constitute the best silicon contribution, but refining, unfortunately, eliminates the envelopes of these products, which is the richest parts in silicon. The biological graminaes (rice, corn, barley, millet, etc.) are thus more interesting. The bamboo, mushrooms, horsetail and fruit coating constitute an appreciable source of silica.

### Bone and Cartilage

It was reported in several studies on animal and human, that bone and cartilage constitute a privileged sector of silicon action (Carlisle, E. M. 1980. A silicon requirement for normal skull formation in chicks. J. Nutr., 110, 352-359; Seaborn, C. D. and Neilson. 2002. Dietary silicon and arginine affect mineral element composition of rat femur and vertebra. Biol. Trace Elem. Res., 89, 239-250). Silicon is a fundamental support agent of cartilage and stimulates its growth. Silicon is part of elastic fibers, collagen fibers and proteoglycan compositions, conferring the physical properties of shape, tonicity and elasticity to cartilage (Schwarz, K. A. 1973. bound form of silicon in glycosaminoglycans and polyuronides. Proc Natl Acad Sci., 70, 1608-1612).

The progressive deprivation results in a loss of flexibility, a process of diffuse sclerosis and excessive calcic deposits. On the other hand, silicon's contribution, stimulating the synthesis of the conjunctive components, contributes to reinforce cartilage and comfort. In bone tissue, silicon controls collagen and proteoglycans synthesis (U.S. Pat. No. 6,335,457). Among animal, a silicon deficiency involves significant growth disorders such as shortening of bones, cranial and articular abnormal formations (Carlisle, E. M. 1980. Biochemical and morphological changes associated with long bone abnormalities in silicon deficiency. J. Nutr., 110, 1046-56). In ovariectomized rats, silicon supply allows to prevent osteoporosis and to gain bone mass compared to non-treated subjects (Seaborn, C. D. and Neilson. 2002. Dietary silicon and arginine affect mineral element composition of rat femur and vertebra. Biol. Trace Elem. Res., 89, 239-250). For human, silicon shortens necessary time for bone fracture repair in a significant way (Schiano A. Eisinger F. Detolle P. Laponche A M. Brisou B. Eisinger J. 1979. Silicon, bone tissue and immunity. Rev. Rhum. Mal. Osteoartic., 46, (7-9), 483-486).

### Muscles Also Benefit of Silicon Supply:

By preventive cures, silicon decreases the frequency of tendinitis and tendinous ruptures, particularly among athletes;

Silica gel has an antalgic action, generally fast and significant, even in persistent tendinitis cases.

### Skin

Skin is naturally rich in silicon. With age, the silicon reserve depletes progressively. Silicon acts on conjunctive structure level ensuring the integrity, tonicity and elasticity of cutaneous tissue by its bonds with collagen, proteoglycans and elastin. A silicon supply has many positive consequences on skin, such as a better hydration and tonicity, an accelerated hair growth, a smooth and firmer skin, and a faster wounds and burn cicatrisation, which alleviates quickly (Oberbaum, M., Markovits, R., Weisman, Z., Kalinkevits, A., Bentwich, Z. 1992. Wound healing by homeopathic silica dilutions in mice. Harefuah, 123, (3-4), 79-82, 156).

Various dermatitis, such as eczema, psoriasis and acne can draw benefit from silica. “Breakable” nails are often improved by silicon uptake (Lassus, A. 1993. Colloidal silicic acid for oral and topical treatment of aged skin, fragile hair and brittle nails in females. J. Int. Med. Res., 21, (4), 209-15).

### Vessels

Silicon is naturally present in arterial walls, ensuring their tonicity and flexibility. Certain epidemiological studies on water showed a reduction of cardiovascular diseases among subjects consuming water rich in silica, notably in Finland (Schwarz, K, Ricci, B. A., Punsar, S., Karvonen, M. Opposite relation of silicon in drinking toilets and arteriosclerosis in Finland. Lancet, 1977, 1, 538-539).

There are several explanations for the actions of silicon on the vascular system, such as silicon being an integral part of the constitutive tissue of vascular walls and silicon preventing the dangerous bonding between circulating lipids and the arterial walls. This was particularly illustrated among rats subjected to hyperlipidic diet and it was shown that silicon contributes to lower cholesterol level.

It is worth to mention that silicon could constitute an antidote to aluminum excess: aluminum hydroxide could appear to be highly toxic (Perez-Granados, A. M., Vaquero, M. P. 2002. Silicon, aluminum, arsenic and lithium: essentiality and human health implications. J. Nutr. Health Aging, 6, (2), 154-162). Implicated in many pathologies (i.e. degenerative neurological diseases as Alzheimer's disease), it is present in aluminum utensils, food wrapping, some vaccines and water in soft drinks.

These data suggest that silicon is considered as a significant supplement for human or, in certain particular cases, used as a treatment. However, the main problem is the silicon assimilation. In spite of its abundance in nature, silicon remains an insoluble mineral and little or not assimilable by the organism. Silicon can be found in food, essentially in alluminosilicic form which shows a slightly assimilable rate. It seems that the biologically active form of silicon must be the soluble form which depends on the number of free hydroxyl groupings (silanol function).

According to Seguin, M-C. and Gueyne, J. (U.S. Pat. No. 6,335,457), the monomeric or oligomeric forms (slightly polymerized) of the orthosilicic acid are able to pass through the intestinal barrier. Moreover, the presence of silanol groups can make it possible to functionalize it in order to create covalent bonds or hydrogen interactions with amides groupings, alcohol and ketones.

The orthosilicic monomeric acid or Si(OH)₄ is relatively stable with lower concentration than 10⁻⁴ M, but with higher concentrations, the molecules of Si(OH)₄ tend to polymerize themselves to give oligomers or polymers of orthosilicic acid (bond siloxane formation). This formation confers to the solution a colloidal aspect or silica gel which is slightly soluble or insoluble in water (biodisponibility is very weak).

According to Dupuis (U.S. Pat. No. 6,592,854), these anionic (carboxylate) or nonionic groups functionalized in silicic acids were known for treating keratin fibers.

U.S. Pat. No. 6,335,457 discloses orthosilicic acid, obtained (by hydrolysis) from synthetic sources, that is complexed with a polypeptide and used only under solid form.

There is therefore a great need for bioavailable or water-soluble silicic acid complexes and derivatives for therapeutic and cosmetic use.

### SUMMARY OF THE INVENTION

One aspect of the present invention is to provide a method for isolating from a natural source a soluble silicon compound of formula:

(R₁)ₙSi(OH)₄₋ₙ

wherein n is an integer from 0 to 3;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
said method comprising the steps of:
heating shavings or powder of said natural source in an aqueous alkaline solution at a temperature below about 150° C.; and
recovering said soluble silicon compound.

In a further aspect, the present invention provides a soluble silicon compound of formula:

(R₁)ₙSi(OR₂)_{q}(OH)_{4-n-q}

wherein n is an integer from 0 to 3, q is an integer from 1 to 4 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein;

In a further aspect, the present invention provides a method for preparing a functionalized soluble silicon compound of formula:

(R₁)ₙSi(OR₂)_{q}(OH)_{4-n-q}

wherein n, q, R₁ and R₂ are as defined herein; said method comprising the step of:
treating an acidic aqueous solution of a compound of formula (R₁)ₙSi(OH)₄₋ₙ with a functionalizing agent of formula R₂—X and optionally in the presence of a catalyst, wherein X is a leaving group; and
recovering said functionalized soluble silicon compound.

In a further aspect, there is provided a method for stabilizing and preventing polymerisation of a soluble silicon compound of formula:

(R₁)ₙSi(OR₂)_{q}(OH)_{4-n-q}

wherein n, q, R₁ and R₂ are as defined herein; said method comprising the step of:
adding a stabilizing agent to said compound; and
homogenizing the resulting mixture.

In still a further aspect, there is provided a method for increasing silicon concentration in a recipient comprising administering a soluble silicon compound of formula:

(R₁)ₙSi(OR₂)_{q}(OH)_{4-n-q}

wherein n, q, R₁ and R₂ are as defined herein.

In still a further aspect, there is provided a method for treating or preventing silicon-deficient induced disease in a individual comprising administering a soluble silicon compound of formula:

(R₁)ₙSi(OR₂)_{q}(OH)_{4-n-q}

wherein n, q, R₁ and R₂ are as defined herein.

In a further aspect, the present invention provides a method for preventing silicon concentration depletion in a recipient comprising administering a soluble silicon compound of formula:

(R₁)ₙSi(OR₂)_{q}(OH)_{4-n-q}

wherein n, q, R₁ and R₂ are as defined herein.

In a further aspect of the invention, there is provided a method for retarding or preventing signs of aging of a recipient comprising applying to the skin a soluble silicon compound of formula:

(R₁)ₙSi(OR₂)_{q}(OH)_{4-n-q}

wherein n, q, R₁ and R₂ are as defined herein.

In another aspect, there is provided a cosmeceutical composition comprising a soluble silicon compound of formula:

(R₁)ₙSi(OR₂)_{q}(OH)_{4-n-q}

and pharmaceutically acceptable carrier, diluent, excipient or a mixture thereof; wherein n, q, R₁ and R₂ are as defined herein.

### DETAILED DESCRIPTION OF THE PREFERRED EMBODIMENT

While the making and using of various embodiments are discussed below, it should be appreciated that the specific embodiments discussed herein are merely illustrative of specific ways of making and using the invention and should not be construed as to limit the scope of the invention.

In one embodiment, compounds of the present invention comprise those wherein the following embodiments are present, either independently or in combination.

In one embodiment of the invention there is provided a method for isolating from a natural source a soluble silicon compound of formula:

(R₁)ₙSi(OH)₄₋ₙ

wherein n is an integer from 0 to 3;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
said method comprising the steps of:
heating shavings or powder of said natural source in an aqueous alkaline solution at a temperature below about 150° C.; and
recovering said soluble silicon compound.

In further embodiments:

n is an integer from 1 to 3;
n is 1 or n is 2 or n is 3.

In one embodiment, the step of recovering is comprising cooling the aqueous alkaline solution and removing insoluble residues.

In a further embodiment, the method is further comprising adjusting the aqueous solution to a pH of from about 2 to about 10 to obtain a precipitate.

In still a further embodiment, the pH is from about 5 to about 8.

In still a further embodiment, the pH is from about 2 to about 6.

In a further embodiment, the method is further comprising washing the precipitate with water.

In one embodiment the natural source is a plant.

In one further embodiment the natural source is bamboo.

In further embodiments:

the temperature is from about 20° C. to about 130° C.;
the temperature is from about 60° C. to about 110° C.;
the temperature is from about 80° C. to about 100° C.;
the temperature is from about 20° C. to about 80° C.

In further embodiments:

the heating is maintained from about 1 minute to about 48 hours;
the heating is maintained from about 30 minute to about 24 hours;
the heating is maintained from about 1 hour to about 10 hours.

In still further embodiments:

R₁ is C₁₋₁₂ alkyl;
R₁ is C₁₋₆ alkyl;
R₁ is C₁₋₃ alkyl;
R₁ is C₁ alkyl;
C₁ alkyl is methyl;
R₁ is C₂ alkyl;
C₂ alkyl is ethyl.

In one embodiment of the invention, there is provided a method for isolating from a natural source a soluble silicon compound of formula:

(R₁)ₙSi(OH)₄₋ₙ

wherein n is an integer from 0 to 3;
R₁ is selected from the group consisting of optionally substituted C₁₋₆ alkyl, wherein each R₁ is same or different when n is 2 or 3;
said method comprising the steps of:
heating shavings or powder of said natural source in an aqueous alkaline solution at a temperature below about 150° C. for a period of from about 1 hour to about 10 hours; and
cooling the aqueous alkaline solution and removing insoluble residues
adjusting the aqueous solution to a pH of from about 4 to about 10 to obtain a precipitate.

In one embodiment, n is an integer from 1 to 3.

In one embodiment, the method is further comprising the step of washing the precipitate.

In one embodiment, the method is further comprising the step of solubilizing the precipitate in an alkaline solution and adjusting the pH to a final pH of 2 to 7.

In one embodiment of the invention, there is provided a method for preparing a functionalized soluble silicon compound of formula:

(R₁)ₙSi(OR₂)_{q}(OH)_{4-n-q}

wherein n is an integer from 0 to 3, q is an integer from 1 to 4 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein;
said method comprising the steps of:
treating an acidic aqueous solution of a compound of formula (R₁)ₙSi(OH)₄₋ₙ with a functionalizing agent of formula R₂—X and optionally in the presence of a catalyst, wherein X is a leaving group; and
recovering said functionalized soluble silicon compound.

In one embodiment, n is an integer from 1 to 3, q is an integer from 1 to 3.

In further embodiments:

n is an integer from 1 to 3;
n is 1 or n is 2 or n is 3.

In further embodiments:

q is an integer from 1 to 3;
q is 1 or n is 2 or n is 3.

In one embodiment, the step of recovering said functionalized soluble silicon compound is comprising cooling the acidic aqueous solution, and adjusting said solution to a pH of between about 1 to about 7.

In a further embodiment, the step of treating the aqueous solution of the compound of formula (R₁)ₙSi(OH)₄₋ₙ with a functionalizing agent is carried at a temperature of from about 20° C. to about 100° C.

In further embodiments:

the temperature is from about 30° C. to about 100° C.;
the temperature is from about 40° C. to about 100° C.;
the temperature is from about 40° C. to about 60° C.;
the temperature is from about 80° C. to about 100° C.

In further embodiments:

the catalyst is a mineral acid or organic acid;
the catalyst is acetic acid, sulphuric acid, nitric acid or hydrochloric acid.

In further embodiments:

the catalyst represent about 0.01%-1% by weight;
the catalyst represent about 0.05%-0.5% by weight.

The catalyst amount % is defined with regard to the total weight of the solution

In further embodiments:

the aqueous solution has a pH below about 6;
the pH of said acidic solution is from about 1 to about 4;
the pH is adjusted to from about 1.5 to about 3.5.

In still further embodiments:

the heating is maintained for a period of from about 1 minute to about 24 hours;
the heating is maintained for a period of from about 30 minutes to about 6 hours;
the heating is maintained for a period of from about 30 minutes to about 4 hours.

In still further embodiments:

the functionalizing agent of formula R₂—X is a C₁₋₁₂ acyl halide, C₁₋₁₂ alkyl halide, epoxyde, C₁₋₁₂ acyl anhydride;
the functionalizing agent of formula R₂—X is a C₂₋₆ acyl halide, C₁₋₆ alkyl halide, epoxyde, C₂₋₆ acyl anhydride;
the functionalizing agent of formula R₂—X is acetylchloride, monochloroacetic acid, succinic anhydride, epichlorohydrin or bromopropanol.

In one embodiment, R₂ is selected from the group consisting of optionally substituted C₁₋₆ alkyl, optionally substituted C₂₋₁₂ acyl and C(O)Ru wherein Ru is the residual portion of an amino acid, a peptide or a protein.

In one embodiment, R₂ is selected from acetyl, carboxymethyl, succinyl, 2-chloroethyl and 3-hydroxypropanol.

In further embodiments:

R₁ is C₁₋₁₂ alkyl;
R₁ is C₁₋₆ alkyl;
R₁ is C₁₋₃ alkyl;
R₁ is C₁ alkyl;
C₁ alkyl is methyl;
R₁ is C₂ alkyl;
C₂ alkyl is ethyl.

In one embodiment of the invention, there is provided a method for preparing a functionalized soluble silicon compound of formula:

(R₁)ₙSi(OR₂)_{q}(OH)_{4-n-q}

wherein n is an integer from 0 to 3, q is an integer from 1 to 4 and the total of q and n is equal or less than 4;
R₁ is selected from the group consisting of optionally substituted C₁₋₆ alkyl, optionally wherein each R₁ is same or different when n is 2 or 3;
R₂ is selected from the group consisting of optionally substituted C₁₋₁₂ alkyl, optionally substituted C₂₋₁₂ acyl and C(O)Ru wherein Ru is the residual portion of an amino acid, a peptide or a protein;
said method comprising the steps of:
heating an acidic aqueous solution of a compound of formula (R₁)ₙSi(OH)₄₋ₙ with a functionalizing agent of formula R₂—X and optionally in the presence of a catalyst, wherein X is a leaving group and the acidic aqueous solution has a pH of from about 1.5 to 4;
cooling the acidic aqueous solution, and
adjusting said solution to a pH of between about 2 to about 7,
   wherein the heating is maintained at about 80° C. to about 100° C. for a period of from about 30 minutes to about 4 hours; R₂—X is a C₁₋₁₂ acyl halide, C₁₋₁₂ alkyl halide, epoxyde, C₁₋₁₂ acyl anhydride or chloride.

In one embodiment of the invention, there is provided a method for stabilizing and preventing polymerisation of a soluble silicon compound of formula:

(R₁)ₙSi(OR₂)_{q}(OH)_{4-n-q}

wherein n is an integer from 0 to 3, q is an integer from 1 to 4 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein;
said method comprising the step of:
adding a stabilizing agent to said compound; and
homogenizing the resulting mixture.

In one embodiment, the homogenization is carried in aqueous mixture.

In further embodiments the method is further comprising:

heating at a temperature of from about 25° C. to about 100° C.;
heating at a temperature of from about 40° C. to about 60° C.

In further embodiments,

the heating is carried over a period of from about 1 minute to about 24 hours;
the heating is maintained from about 15 minute to about 6 hours;
the heating is maintained from about 30 minutes to about 3 hours.

In still further embodiments,

the stabilizing agent is an amino acid, a protein, a saccharide, a polymer or a mixture thereof;
the stabilizing agent is an amino acid selected from the group consisting of proline, glycine, lysine and a mixture thereof;
the stabilizing agent is an protein selected from the group consisting of gelatin, collagen or hydrolyzed collagen and a mixture thereof;
the stabilizing agent is a polymer selected from the group consisting of polyethylene glycol, polypropylene glycol, polyvinylpyrollidone and a mixture thereof.

In one embodiment, the saccharide, is a polysaccharide.

In a further embodiment, the polysaccharide is selected from the group consisting of alginate, hyaluronate, agar and mixture thereof.

In one embodiment of the invention, there is provided a method for stabilizing and preventing polymerization of a soluble silicon compound of formula:

(R₁)ₙSi(OR₂)_{q}(OH)_{4-n-q}

wherein n is an integer from 0 to 3, q is an integer from 1 to 4 and the total of q and n is equal or less than 4;
R₁ is selected from the group consisting of optionally substituted C₁₋₆ alkyl, optionally wherein each R₁ is same or different when n is 2;
R₂ is selected from the group consisting of optionally substituted C₁₋₁₂ alkyl, optionally substituted C₂₋₁₂ acyl and C(O)Ru wherein Ru is the residual portion of an amino acid, a peptide or a protein;
said method comprising the step of:
adding a stabilizing agent to said compound in aqueous mixture;
homogenizing the resulting mixture; and
heating the mixture at a temperature of from about 40° C. to about 60° C. for a period of from about 30 minutes to about 3 hours.

In one embodiment of the invention, there is provided a method for increasing silicon concentration in a patient comprising administering a soluble silicon compound of formula:

(R₁)ₙSi(OR₂)_{q}(OH)_{4-n-q}

wherein n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid a peptide or a protein.

In one embodiment of the invention, there is provided a method for treating or preventing silicon-deficient induced disease in a patient comprising administering a soluble silicon compound of formula:

(R₁)ₙSi(OR₂)_{q}(OH)_{4-n-q}

n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein.

In one embodiment of the invention, there is provided a method for preventing silicon concentration depletion in a patient comprising administering a soluble silicon compound of formula:

(R₁)ₙSi(OR₂)_{q}(OH)_{4-n-q}

n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein.

In one embodiment of the invention, there is provided a method for retarding or preventing signs of aging of a recipient comprising applying to the skin a soluble silicon compound of formula:

(R₁)ₙSi(OR₂)_{q}(OH)_{4-n-q}

n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein.

In one embodiment of the invention, there is provided a method for whitening skin of a recipient comprising applying to the skin a soluble silicon compound of formula:

(R₁)ₙSi(OR₂)_{q}(OH)_{4-n-q}

n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid a peptide or a protein.

In one embodiment of the invention, there is provided a cosmeceutical composition comprising a soluble silicon compound of formula:

(R₁)ₙSi(OR₂)_{q}(OH)_{4-n-q}

and pharmaceutically acceptable carrier, diluent, excipient or a mixture thereof;
n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid a peptide or a protein.

In one embodiment of the invention, there is provided a soluble silicon compound of formula:

(R₁)ₙSi(OR₂)_{q}(OH)_{4-n-q}

n is an integer from 1 to 3, q is an integer from 1 to 3 and the total of q and n is equal or less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of optionally substituted C₁₋₁₂ alkyl, optionally substituted C₇₋₁₆ aralkyl, optionally substituted C₂₋₁₂ acyl, optionally substituted C₁₋₁₂ acyloxy, phosphate group, C(O)Ru wherein Ru is the residual portion of an amino acid, a peptide or a protein.

In one embodiment, there is provided a composition comprising a soluble silicon compound of the invention and a pharmaceutically acceptable excipient.

In one embodiment, the silicon composition is further comprising one or more therapeutically effective compound.

It has been observed that the orthosilicic acid and its derivatives in solution are relatively stable until a concentration of about 1 mg/100 mL. Without being bound by theory, when the concentration increases, the silicic acids have a tendency to make oligomers and polymers (due to the siloxane bonds formation) and precipitate in solution, which decrease the bioavailability or biodisponibility. In this context, the addition of stabilizers has been found to prevent or reduce this polymerization phenomenon and allows preserving the assimilable monomeric shape of these acids.

In one aspect, the present invention provides a soluble complex obtained by interactions hydrogen or by functionalization, which contained organic silicic acids extracted from plants (i.e. bamboo) using proteins as stabilizers. This soluble complex of organic silicic acids is used under liquid form for therapeutic or cosmetic applications.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

For the purpose of the present invention the following terms are defined below.

The term “alkyl” represents a linear, branched or cyclic hydrocarbon moiety having 1 to 12 carbon atoms, which may have one or more double bonds or triple bonds in the chain, and is optionally substituted. Examples include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, hexynyl, cyclopropyl, cyclobutyl, cyclohexenyl, cyclohex-dienyl and cyclohexyl. The term alkyl is also meant to include alkyls in which one or more hydrogen atom is replaced by a halogen, ie. an alkylhalide. Examples include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl.

The term “aralkyl” represents an aryl group attached to the adjacent atom by a C₁₋₆ alkyl Examples include but are not limited to benzyl, benzhydryl, trityl, phenethyl, 3-phenylpropyl, 2-phenylpropyl, 4-phenylbutyl and naphthylmethyl.

The term “aryl” represents a carbocyclic moiety containing at least one benzenoid-type ring (i.e. may be monocyclic or polycyclic), and which may be optionally substituted with one or more substituents. Examples include but is not limited to phenyl, tolyl, dimethyphenyl, aminophenyl, anilinyl, naphthyl, anthryl, phenanthryl or biphenyl.

The term “acyl” is defined as a radical derived from a carboxylic acid, obtained by replacement of the —OH group. Like the acid to which it is related, an acyl radical may be straight chain, branched chain or cyclic aliphatic or aromatic. Examples include but are not limited to formyl, acetyl, propionyl, butyryl, isobutyryl, valeryl, isovaleryl, pivaloyl, caproyl, isocaproyl, acryloyl, propioloyl, methacryloyl, crotonoyl, isocrotonoyl, benzoyl, naphthoyl, toluoyl, cinnamoyl, furoyl, glyceroyl, salicyloyl.

The term “phosphate” represents a derivative of phosphoric acid and is meant to include salts, esters and amide of phosphoric acid and may be defined by the formulas:

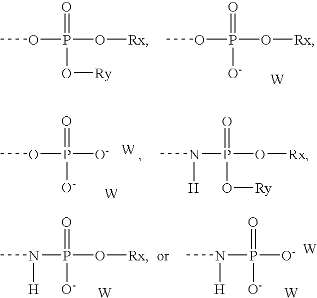

wherein Rx and Ry are each independently hydrogen or C₁₋₆ alkyl;
or Rx and Ry are taken together with the oxygen to which they are attached to form a 5 to 7 membered heterocycle; and
W has one or two positive charge and is alkali metal, alkaline earth metal or ammonium.

The term “heterocycle” represents an optionally substituted saturated, unsaturated or aromatic cyclic moiety wherein said cyclic moeity is interrupted by at least one heteroatom selected from oxygen (O), sulfur (S) or nitrogen (N). Heterocycles may be monocyclic or polycyclic rings. Examples include but are not limited to azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl and thiopyranyl, furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl.

The term “optionally substituted” or “optional substituent” represents one or more halogen, amino, amidino, amido, azido, cyano, guanido, hydroxyl, nitro, nitroso, urea, OS(O)₂Rm (wherein Rm is selected from C₁₋₆ alkyl, C₆₋₁₀ aryl or 3-10 membered heterocycle), OS(O)₂ORn (wherein Rn is selected from H, C₁₋₆ alkyl, C₆₋₁₀ aryl or 3-10 membered heterocycle), S(O)₂ORp (wherein Rp is selected from H, C₁₋₆ alkyl, C₆₋₁₀ aryl and 3-10 membered heterocycle), S(O)₀₋₂Rq (wherein Rq is selected from H, C₁₋₆ alkyl, C₆₋₁₀ aryl or 3-10 membered heterocycle), OP(O)ORsORt, P(O)ORsORt (wherein Rs and Rt are each independently selected from H or C₁₋₆ alkyl), C₁₋₆ alkyl, C₆₋₁₂ aralkyl, C₆₋₁₀ aryl, C₁₋₆ alkoxy, C₆₋₁₂ aralkyloxy, C₆₋₁₀ aryloxy, 3-10 membered heterocycle, C(O)Ru (wherein Ru is selected from H, C₁₋₆ alkyl, C₆₋₁₀ aryl, C₆₋₁₂ aralkyl or 3-10 membered heterocycle), C(O)ORv (wherein Rv is selected from H, C₁₋₆ alkyl, C₆₋₁₀ aryl, C₆₋₁₂ aralkyl or 3-10 membered heterocycle), NRxC(O)Rw (wherein Rx is H or C₁₋₆ alkyl and Rw is selected from H, C₁₋₆ alkyl, C₆₋₁₀ aryl, C₆₋₁₂ aralkyl or 3-10 membered heterocycle, or Rx and Rw are taken together with the atoms to which they are attached to form a 3 to 10 membered heterocycle) or SO₂NRyRz (wherein Ry and Rz are each independently selected from H, C₁₋₆ alkyl, C₆₋₁₀ aryl, C₃₋₁₀ heterocycle or C₆₋₁₂ aralkyl).

As used herein, the term “recipient” is taken to mean warm blooded animals such as mammals, for example, cats, dogs, mice, guinea pigs, horses, bovine cows, sheep, or humans; preferably the recipient is a human. The recipient may be healthy, in a diseased state or at risk of being in a diseased state and susceptible to benefit from the administration of a soluble silicon compound whether it is provided orally, topically or otherwise.

As used herein, the terms “bioavailability” and “biodisponibility” are used interchangeably to denote the absorbable characteristic of organic silicic acids when administered orally.

As used herein, the term “mineral acid” is an acid obtained from minerals. Examples include but is not limited to hydrochloric acid, sulphuric acid, nitric acid, phosphoric acid, hydrofluoric acid, chromic acid and boric acid This term is also referred to as inorganic acid by the skilled person in the art.

As used herein, the term “organic acid” is acid containing carbon. Examples include but is not limited to carboxylic acids (—COOH) such as formic acid, acetic acid, trifluoroacetic acid, and citric acid, sulfonic acids (—SO3H) such as methanesulfonic acid and trifluoromethanesulfonic acid.

As used herein, the term “therapeutic agent” refers to an agent that has activity in a biological system. Particularly useful classes of therapeutic agent include, but are not limited to, cough suppressants, such as dextromethorphan hydrobromide and codeine; antibiotics such as cephalosporin; antihistamines such as chlorpheniramine maleate, brompheniramine maleate, loratidine, astemizole, diclofenac sodium and terfenadine; decongestants such as pseudoephedrine and phenylephrine; antihypertensives such as ACE-inhibitors, verapamil, nifedipine, propanolol, metoprolol, metoprolol succinate, metoprolol fumarate, metoprolol, methylphenadate, tartarate; agents to treat attention deficit disorder/hyperactivity such as methylphenadate, d and/or l isomers of methylphenadate, amphetamines, d and/or l isomers of amphetamines, and combinations of amphetamines; calcium channel blockers such as verapamil, diltiazam, nifedipine, nimodipine, felodipine, nicardipine, isradipine and amlodipine; antidiabetic agents such as glipizide and ibromectin; proton pump inhibitors such as omeprazole; anti-convulsants and anti-epileptics such as valproate sodium, clonazepam, gabapetin, and topiramate; anti-depressives such as buspirone, fluoxeline, 5-hydroxytryptamine receptor agonist and antagonist; anti-migraines such as sumatreptan and dihydroergotamine; antipsychotics such as resperidone; antiemetics such as ondansetron; anti-heartburns such as cisapride; H₂ receptor antagonists such as cimetidine, ranitidine, famotidine, nizatidine; carbamazepine; beta adrenergic receptor blockers; anti-Parkinson agents such as selegiline, carbidopa/levodopa, pergolide, bromocriptine, amantadine, trihexyphenidyl HCl; antiviral agents including antiherpesvirus agents such as acyclovir, famciclovir, valcyclovir, foscamet, ganciclovir; antiretroviral agents such as didanosine, stavudine, zalcitabine, zidovudine; and others such as amantadine, interferon alpha, ribavirin, rimantadine; anti Alzheimer's agents such as galantamine; and other therapeutic agents such as cimetidine, propiomazine, phenyloin, tacrine, propiazam, proplazam; vinca alkaloid.

The carrier(s), diluent(s) or excipient(s) must be “acceptable” in the sense of being compatible with the other ingredients of the formulation and not being deleterious to the recipient thereof.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 60 mg/kg of body weight per day, alternatively in the range of 0.5 to 6 mg/kg/day, in a further alternative in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 1 to 1500 mg, as a further example the unit dosage form is containing 10 to 1000 mg, as a further example the unit dosage form is containing 50 to 750 mg of active ingredient per unit dosage form.

Ideally the active ingredient should be administered to achieve peak plasma concentrations of the active compound. This may be achieved, for example, by the intravenous injection of a solution of the active ingredient, optionally in saline, or orally administered as a bolus. Desirable blood levels may be maintained by a continuous infusion or by intermittent infusions.

While it is possible that, for use in therapy, a compound or combination of the invention may be administered as the raw chemical it is preferable to present the active ingredient as a pharmaceutical composition.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds and combinations according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

For topical administration to the epidermis, the compounds and combinations according to the invention may be formulated as ointments, gel, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

The organic silicic acid compounds are maintained stable by hydroxyl groups, which insure a hydration action. However, to improve the tonicity and protection effects, the addition of proteins such as gelatin and/or collagen (stabilizers) seems necessary. Polysaccharides such as pectin, chitosan and hyaluronan, etc. (thickener and cytostimulating) and antioxidant agents such as tocopherol, ascorbic acid and polyphenols, etc. (antiradical) is also possible. Furthermore, the addition of proteins such as collagen, gelatin or whey protein, is advantageous for their skin regeneration activities.

As silicon is an essential mineral required for the strength and elasticity of tissue, hair, skin, nails, etc., the addition of antioxidants (e.g. vitamin E, ascorbic acid and polyphenols, etc.) and cytostimulating compounds (e.g. collagen, gelatin, vitamin C, etc.) allow to improve the skin protection and treatment.

Arbutin, a glycosylated hydroquinone, acts as a depigmenting agent (skin whitening agent) as it inhibits melanin synthesis by inhibition of tyrosinase activity. The combination of arbutin or its derivative (hydroquinones) and organic silicic acid is of interest for treatment or protection against free radical damages (from sun and smoking).

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

When desired the above described formulations adapted to give sustained release of the active ingredient may be employed.

The present invention will be more readily understood by referring to the following examples which are given to illustrate the invention rather than to limit its scope.

### EXAMPLES

The extraction of organic silicic acids generally consist in heating the shavings or powder of a convenient source (i.e. bamboo, alga or horse tail) in an aqueous alkaline solution, which can be prepared from an alkali-hydroxide (for example sodium hydroxide or potassium hydroxide) to produce an extract. The pH of this extract is reduced to about pH 10.0 (to eliminate the lignine for example using an acid such as sulfuric, chlorhyric or phosphoric acids) and the extract is then filtered to eliminate the cellulose and others insoluble residues. Thereafter, the filtrate was adjusted at pH 5.0-8.0 (preferably organic acid such as lactic acid) to provide a white precipitate containing silicic acids and its derivatives in solution, which was washed several times to eliminate the salts.

### Example I

### Extraction of Organic Silicic Acids from Bamboo

Organic silicic acids is obtained by extraction from the bamboo (i.e. Bambusa vulgaris rich in organic silica) and particularly, parts of the nodes of the stems of bamboo, also called “bamboosil” or “to tabashir”. Indeed, an amount of 600 g of bamboo powder was treated in 400 g of 25% NaOH solution (pH>12.0) and the mixture was heated between 90-100° C. for minimum 2 h. After heating, the mixture was filtered to eliminate the insoluble residues and the pH of extract was reduced at 10 using HCl (1-10% to eliminate the lignine). Thereafter, the extract liquid was filtered once more time and treated with lactic or citric acid until a light fluffy precipitate formed (pH 5.0-8.0), which was separated by filtration. The obtained precipitate was washed several times with distilled water (to eliminate salts and other residues), which is recovered as organic silicic acid and its derivatives by drying.

### Preparation of Organic Silicic Acids Solution

To obtain the soluble organic silicic acids, an amount of 2.0-3.0 g of the precipitate obtained previously was redispersed in 50 mL of NaOH (0.5-2.0 M, pH >10.0) solution, which was heated at 90-100° C. during 30-120 min. Thereafter, the solution was filtered and the filtrate was completed with 100 mL of citric acid (6-12%) solution containing approximatively 3.0% of gelatin or collagen (preferably fish gelatin, i.e. gelatin of Nitta Gelatin type FGL-200TS). The final pH value was between 3.2-4.8. This solution, slightly white and translucent, contains essentially the soluble organic silicic acids that are stable for several weeks or months depending of the concentration of organic silicic acids. (In the above process, citric acid could also be replaced by acetic acid, lactic acid, propanoic acid, etc.)

The concentration of the silicic acids was determined spectrophotometrically by measuring the absorbance of a molybdenum complex at 640 nm as described in Bunting, W. E. (1944), The determination of soluble silica in very low concentrations: Industrial and engineering chemistry. Analytical Ed. vol. 16, pp. 612-615). Specifically, a volume of 0.5 mL of silicic solution (obtained previously) was dispersed in polyethylene or teflon beaker containing mL of the molydate solution 0.6%. If the silicic concentration is low, larger aliquot of sample solution should be used. The mixture was stirred for 10 minutes and a volume of 25 mL of reducing solution comprising 0.028% of sodium sulfite, 0.36% of sodium bisulfite, and 0.006% of 1-amino-2-naphtol-4-sulfonic acid was added. To avoid the interference can cause by phosphate, a volume of 25 mL of tartaric acid (1.6%) was introduced in the mixture to destroy the phosphomolydic acid complex. The mixture was stirred for 45 minutes and the absorbances were read at 640 nm. The color of the complex is stable for at least 6 h and the silicon (SiO₂) was used for the calibration.

### Example II

### Extract of Soluble Organic Silicic Acids from Other Sources

Another manner could be used to obtain the organic silicic acids using the powder provided by Draca Natural Product inc. (San Jose, Calif., USA), which is a bamboo extract containing more than 65% of organic silica. The preparation consists in washing several times 400 g of bamboo powder with distilled water until obtaining a slightly white powder. These powders are then dispersed in NaOH solution (20-25%, pH >12.0) for a final volume of 1 L which was heated between 80-100° C. during 1.0-2.0 h. The mixture was then filtered and the filtrate was conserved in a porcelain bottle or container. The silica concentration in the filtrate is approximatively 30%.

To obtain the silicic acid suspension, an amount of 3-6 mL of filtrate obtained previously was added in 94-97 mL of citric acid (3.0-6.0%) (or acetic acid, lactic acid, propanoic acid, etc.) solution containing approximatively 3.0% of gelatin or collagen.

It is worth to note that the increase of silicic acid concentration more than 1.5% is possible. In this case, the gelatin or collagen is equally increased and the ratio silicic acid:protein is 1:3.5.

### Example III

### Stabilization by Gelification of Organic Silicic Acids Using a High Concentration of Neutral Protein (Gelatin or Collagen)

An amount of 3-6 mL of silica extract (filtrate obtained previously) was added in 94-97 mL of citric acid (3.0-6.0%) solution containing 4.0-5.0% of gelatin (or collagen). The characteristic of this solution was that it remained in the liquid form at room temperature (about 22° C.), and became in the gel form at a temperature lower than 16° C. Consequently, the stability of silicic acid can be improved for extended period of time (likely several years) if it is conserved at low temperatures.

### Example IV

### Improvement of Conservation Time of Organic Silicic Acids by Functionalization: Succinylation

An amount of 5-10 g of succinic anhydride was added in 100 mL of silica extract (obtained previously in example I or II, pH about of 11.0) and heated at 60-70° C. during 1-2 h. Thereafter, the matter was precipitated at pH 5.0-7.0 using hydrochloride acid and washed several times with methanol then water to eliminate the secondary products (i.e. succinic acid formed between hydroxyl of water and succinic anhydride). The precipitate was dissolved in NaOH solution and the silicic acid solution was obtained using the similar process described previously in example III. The silicic acid solution was slightly white and translucent. The accelerated stability study showed that the functionalized silicic acid remained in solution for a period longer than those without functionalization (approximatively two times).

### Example V

### Improvement of Conservation Time of Organic Silicic Acids by Functionalization: Acetylation

Acetylation process consists in connecting on the silicic acids an acetyl group by acetylation in presence of a catalyst (e.g. acetic acid, sulfuric acid or hydrochloric acid, etc.).

A volume of 100 mL of silica extract (obtained previously in example I or II, pH about of 11.0) was added in 15-20 mL of acetic anhydride solution and heated at 70-90° C. during 1-2 h. Thereafter, the matter was precipitated at pH 5.0-7.0 using NaOH and washed several times with methanol then water to eliminate the secondary products (i.e. acetic acid formed between hydroxyl of water and succinic anhydride). After filtration, the precipitate was dissolved in NaOH solution and the functionalized silicic acid solution was obtained using the similar process described previously in example III. The silicic acid solution was slightly white and translucent.

The acetylation process could be synthesized as described previously in acid media at pH 1.5-3.0. The volume of anhydride acetic (15-20 mL) was added dropwise in the solution for 1-2 h at 70-90° C.

The stability test showed that the functionalized silicic acid so obtained benefited of improved stability with regard to unfunctionalized silicic acid, the stability was less than that obtained from succinyl silicic acid.

The functionalized silicic acid obtained in example IV and V is characterized by FTIR, after precipitation and washings to remove side products, through identification of the C═O band at 1700-1750 cm⁻¹ and for succinyl silicic acid, a carboxylate band at 1400-1450 cm⁻¹.

### Example VI

### Organic Silica Formulation for <<Skin-Nail-Hair> Care

An amount of 0.5% of litchi extract (From Litchi chinensis), 1.2% of Yudzu extract (Citrus junos), and 2.0% of polyphenols extract (green tea) were added in 50 mL of purified water under stirring. After homogenization, a volume of 50 mL of silicic acid solution (2.0-2.6%) was added in the mixture.

Other additives could be also added in the silicic acid for different applications such as glucosamine, chondroitine, collagen (for <<joint formulation>>), isoflavones, calcium, magnesium (for bone health formulation>) or other supplements such as vitamins and minerals (zinc, selenium, cooper, etc.).

### Example VII

### Beads or Microbeads Formulation

The solution obtained herein is formulated as beads or microbeads by the addition, in the silicic solution, of an appropriate amount of a polysaccharide.

A typical example is illustrated as follow: silicic based-beads or microbeads were obtained by dropping or by atomization of the organic silicic acid solution (obtained as described in Example I, II or III) or functionalizing silicic acid (obtained as described in Example IV or V) containing 0.5-3.0% of alginate in the gelling solution (e.g. 1-10% of calcium chloride).

Another method for product beads or microbeads is by gelification of gelatin or collagen. For this purpose, the increase of gelatin (or collagen) concentration in the formulation is necessary and the preparation is as described in example III.

An amount of 3-6 mL of silica extract (filtrate obtained previously) was added in 94-97 mL of citric acid (3.0-6.0%) solution containing 4.0-5.0% of gelatin (or collagen) at 60° C. The latter was added dropwise in the oil (i.e. canola oil) solution at 5-10° C. under mild stirring until beads became solid. For the microbeads, a volume of 1 mL of silicic acid was introduced in 40 mL of oil solution (5-10° C.) under strong stirring. Thereafter, the microbeads could be isolated by sedimentation or by slight centrifugation.

### Example VIII

### Preparation of Tablets

The organic silicic solution (obtained as described in Examples I, II, III, IV and V) was dried by casting at room temperature during 24-48 h depending on the volume. The formed films was broken and blended in powder which was sieved to obtain the powder inferior to 50 meshes. Tablets of 100 mg was obtained by direct compression (2.3 T/cm2, Carver Hydraulic Press) of the Methocel (cellulose derivatives) as matrix containing 1-10% of powders of organic silicic acid-gelatin complex.

### Example IX

### Preparation of Organic Silicic Acid Based Gel for Cosmetic Applications

Gel based formulation was obtained by treatment of the bamboo powders (15-20 g) in 0.5 L of NaOH (5-10 M) solution at 100° C. After heating during 1-3 h, the solution was filtered to eliminate the insoluble residues (cellulose and others). Thereafter, the organic silica extract (filtrate) was neutralized with a hydrochloride acid solution under stirring (the pH optimal varied between 4.0-7.0). The addition of gelatin or collagen is optional (0.5-2.0%). The gel formation was observed after 15 minutes or more at room temperature and the obtained gel was drained to eliminate the water and triturated with a blender in order to obtain a gel suspension.

### Example XII

### Soluble Organic Silicic Acid Based-Cream for Hair Care or Body Care

### Aqueous Phase Preparation

A volume of 40 mL of organic silicic acid gel suspension (1.0-2.0% obtained previously in Example XI) was mixed in 40 mL of hyaluronate (0.5-1.0%) and in 2.5-3.5% of glycerol with strong stirring at 40-60° C. It's worth to mention that it's also possible to use the organic silicic acid solution obtained in Example II, III and IV or the powder in Example VI for this preparation. In this case, the viscosity of cream is low and the increase of hyaluronate is necessary (>1.0%).

### Oily Phase Preparation

At the same time, an amount of 4.0 g of stearic acid, 4.0 g of polawax (emulsifying wax NF) and 2.0 g of cetyl alcohol NF were heated at 60-80° C. in the mixture of 1.0 mL of linseed oil, 2.0 mL of vitamin E and 2.0 mL of mineral oil. When the oily solution was completely melted, the mixture was cooled at 40-50° C. and a volume of 1.5 mL of rosemary essential oil was added with stirring.

In a separated beaker, the oil phase and the aqueous phase was combined with stirring until the mixture is cooled down and became consistency.

### Example XIII

### Soluble Organic Silicic Acid Based-Cream with Anti-Aging Activity

The cream based formulation was prepared according to the example XI with the following composition:

Aqueous phase preparation

| | |
|---|---|
| Silica gel suspension | 40.0-50.0% |
| (or solution obtained in Example IV) | |
| Distilled water | 3.0-7.0% |
| Glycerol | 1.0-2.5% |
| Hyaluronate | 0.5-2.5% |

Oily phase preparation

| | |
|---|---|
| Tocopherol | 1.0-5.0% |
| Linseed oil | 1.0-2.5% |
| Shea butter | 1.0-2.5% |
| Stearic acid | 1.0-5.0% |
| Emulsifying wax NF | 1.0-5.0% |
| Cetyl alcohol NF | 1.0-4.0% |
| Lecithin (or Tween 20-100, optional) | 2.0-10.0% |
| Coenzyme 10 (CoQ10) | 1.0-4.0% |

The preparation was carried out the same manner as described in example 10.

### Example XIV

### Soluble Organic Silicic Acid-Based Cream with Whitening Activity

The cream based formulation was prepared according to the example XI with the following composition:

Aqueous phase preparation

| | |
|---|---|
| Silica gel suspension | 40.0-50.0% |
| (or solution obtained in Example IV) | |
| Distilled water | 5.0-10.0% |
| Glycerol | 1.0-3.0% |
| Hyaluronate | 0.5-2.5% |
| Xanthan gum (optional) | 0.5-2.5% |
| Arbutin | 2.0-3.0% |

Oily phase preparation

| | |
|---|---|
| Tocopherol | 1.0-5.0% |
| Cetyl alcohol NF | 1.0-4.0% |
| Shea butter | 1.0-4.0% |
| Linseed oil | 1.0-2.0% |
| Lecithin (or Tween 20-100, optional) | 2.0-10.0% |

The preparation was carried out the same manner as described in example XI, but an amount of 0.2-4.0% of salicylic acid (dissolved in minimum quantity of ethanol) was added to activate arbutin.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1-91. (canceled)

92. A method for isolating from a natural source a soluble silicon compound of formula:
(R₁)ₙSi(OH)₄₋ₙ
wherein n is an integer from 0 to 3;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
said method comprising the steps of:
heating shavings or powder of said natural source in an aqueous alkaline solution at a temperature below about 150° C.; and
recovering said soluble silicon compound.

93. The method as defined in claim 92, wherein n is an integer from 1 to 3.

94. The method as defined in claim 92, wherein the step of recovering is comprising cooling the aqueous alkaline solution and removing insoluble residues.

95. The method as defined in claim 94, further comprising adjusting the aqueous solution to a pH of from about 2 to about 10 to obtain a precipitate.

96. A method for preparing a functionalized soluble silicon compound of formula:
(R₁)ₙSi(OR₂)_{q}(OH)_{4-n-q}
wherein n is an integer from 0 to 3, q is an integer from 1 to 3 and the total of q and n is less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2 or 3;
R₂ is selected from the group consisting of substituted C₁₋₁₂ alkyl, substituted C₇₋₁₆ aralkyl, substituted C₂₋₁₂ acyl, substituted C₁₋₁₂ acyloxy, phosphate group, C(O)Ru wherein Ru is the residual portion of an amino acid, a peptide and a protein,
wherein the substituent of said alkyl, aralkyl, acyl and acyloxy is carboxyl or amine;
said method comprising the steps of:
treating an acidic aqueous solution of a compound of formula (R₁)ₙSi(OH)₄₋ₙ with a functionalizing agent of formula R₂—X and optionally in the presence of a catalyst, wherein X is a leaving group; and
recovering said functionalized soluble silicon compound.

97. The method as defined in claim 96, wherein n is an integer from 1 to 3, q is an integer from 1 to 3.

98. The method as defined in claim 96, wherein the step of recovering said functionalized soluble silicon compound is comprising cooling the acidic aqueous solution, and adjusting said solution to a pH of between about 1 to about 7.

99. The method as defined in claim 96, wherein the functionalizing agent of formula R₂—X is selected from the group consisting of C₁₋₁₂ acyl halide, C₁₋₁₂ alkyl halide, epoxyde, C₁₋₁₂ acyl anhydride, and mixtures thereof.

100. The method as defined in claim 96, wherein the functionalizing agent of formula R₂—X is selected from the group consisting of C₂₋₆ acyl halide, C₁₋₆ alkyl halide, epoxyde, C₂₋₆ acyl anhydride, and mixtures thereof.

101. The method as defined in claim 96, wherein the functionalizing agent of formula R₂—X is selected from the group consisting of acetylchloride, monochloroacetic acid, succinic anhydride, epichlorohydrin or bromopropanol, and mixtures thereof.

102. The method as defined in claim 96, wherein R₂ is selected from the group consisting of optionally substituted C₁₋₆ alkyl, optionally substituted C₂₋₁₂ acyl and C(O)Ru wherein Ru is the residual portion of an amino acid, a peptide or a protein, and mixtures thereof.

103. The method as defined in claim 96, wherein R₂ is selected from the group consisting of acetyl, carboxymethyl, succinyl, 2-chloroethyl, 3-hydroxypropanol, and mixtures thereof.

104. The method as defined in claim 96, wherein R₁ is C₁₋₃ alkyl.

105. A method for preventing silicon concentration depletion in a recipient comprising administering a soluble silicon compound of formula:
(R₁)ₙSi(OR₂)_{q}(OH)_{4-n-q}
wherein n is an integer from 0 to 3, q is an integer from 1 to 3 and the total of q and n is less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of substituted C₁₋₁₂ alkyl, substituted C₇₋₁₆ aralkyl, substituted C₂₋₁₂ acyl, substituted C₁₋₁₂ acyloxy, phosphate group, C(O)Ru wherein Ru is the residual portion of an amino acid, a peptide and a protein,
wherein the substituent of said alkyl, aralkyl, acyl and acyloxy is carboxyl or amine;
wherein a stabilizing agent selected from the group consisting of gelatin, collagen or hydrolyzed collagen and a mixture thereof is in admixture with said soluble silicon compound.

106. The method as defined in claim 105, wherein n is an integer from 1 to 3, q is an integer from 1 to 3.

107. A method for providing care to skin, hair or nail of a recipient comprising applying a soluble silicon compound of formula:
(R₁)ₙSi(OR₂)_{q}(OH)_{4-n-q}
wherein n is an integer from 0 to 3, q is an integer from 1 to 3 and the total of q and n is less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein each R₁ is same or different when n is 2;
R₂ is selected from the group consisting of substituted C₁₋₁₂ alkyl, substituted C₇₋₁₆ aralkyl, substituted C₂₋₁₂ acyl, substituted C₁₋₁₂ acyloxy, phosphate group, C(O)Ru wherein Ru is the residual portion of an amino acid, a peptide and a protein,
wherein the substituent of said alkyl, aralkyl, acyl and acyloxy is carboxyl or amine;
wherein a stabilizing agent selected from the group consisting of gelatin, collagen or hydrolyzed collagen and a mixture thereof is in admixture with said soluble silicon compound.

108. The method as defined in claim 107, wherein n is an integer from 1 to 3, q is an integer from 1 to 3.

109. A soluble silicon compound of formula:
(R₁)ₙSi(OR₂)_{q}(OH)_{4-n-q}
wherein n is an integer from 0 to 3, q is an integer from 1 to 3 and the total of q and n is less than 4;
R₁ is selected from the group consisting of optionally substituted C₁₋₁₂ alkyl, optionally substituted C₇₋₁₆ aralkyl, optionally substituted C₆₋₁₀ aryl and optionally substituted heterocycle having 3-6 atom members, wherein R₁ is same or different when n is 2:
R₂ is selected from the group consisting of substituted C₁₋₁₂ alkyl, substituted C₇₋₁₆ aralkyl, substituted C₂₋₁₂ acyl, substituted C₁₋₁₂ acyloxy, phosphate group, C(O)Ru wherein Ru is the residual portion of an amino acid, a peptide and a protein,
wherein the substituent of said alkyl, aralkyl, acyl and acyloxy is carboxyl or amine.

110. The compound as defined in claim 109, wherein n is an integer from 1 to 3, q is an integer from 1 to 3.

111. A composition comprising a soluble silicon compound as defined in claim 109 and a pharmaceutically acceptable excipient.
